(19) United States
(12) Patent Application Publication
(10) Pub. No.: US 2023/0286953 A1
(43) Pub. Date: Sep. 14, 2023

(54) METHODS AND INTERMEDIATES FOR PREPARING JAK INHIBITORS

(71) Applicant: Concert Pharmaceuticals, Inc., Lexington, MA (US)

(72) Inventors: Patrick Bazinet, Somerville, MA (US); Robert S. Lewis, Lexington, MA (US); Mahender Karla, Lexington, MA (US); Yong Dong, Chestnut Hill, MA (US)

(21) Appl. No.: 18/013,380

(22) PCT Filed: Jun. 29, 2021
(86) PCT No.: PCT/US21/39653

§ 371 Date: Dec. 28, 2022

Related U.S. Application Data
(60) Provisional application No. 63/045,627, filed on Jun. 29, 2020.

(51) Int. Cl. C07D 403/04; C07D 231/12

(52) U.S. Cl. CPC C07D 403/04; C07D 231/12

(57) ABSTRACT

Improved processes and intermediates for preparing ruxolitinib, deuterated analogs of ruxolitinib, and other JAK inhibitors are disclosed.

### CROSS-REFERENCE TO RELATED APPLICATIONS

This application claims the benefit of U.S. Provisional Patent Application No. 63/045,627, filed on Jun. 29, 2020. The entire teachings of the above application are incorporated herein by reference.

### BACKGROUND OF THE INVENTION

Ruxolitinib phosphate is a heteroaryl-substituted pyrrolo[2,3-d]pyrimidine, also known as 3(R)-cyclopentyl-3-[4-(7H-pyrrolo[2,3-d]pyrimidin-4-yl)-1H-pyrazol-1-yl]propanenitrile phosphate, and as (R)-3-(4-(7H-pyrrolo[2,3-d]pyrimidin-4-yl)-1H-pyrazol-1-yl)-3-cyclopentylpropanenitrile phosphate, which inhibits Janus Associated Kinases (JAKs) JAK1 and JAK2. These kinases mediate the signaling of a number of cytokines and growth factors important for hematopoiesis and immune function. JAK signaling involves recruitment of STATs (signal transducers and activators of transcription) to cytokine receptors, activation and subsequent localization of STATs to the nucleus leading to modulation of gene expression.

Ruxolitinib phosphate has been approved in the US and Europe for the treatment of myelofibrosis and for the treatment of polycythemia vera. Ruxolitinib is currently in clinical trials for the treatment of graft-versus-host disease and other conditions.

A deuterated analog of ruxolitinib phosphate (referred to herein as CTP-543 or Compound (I)) is currently in clinical trials for the treatment of alopecia areata.

Because of the beneficial activities of ruxolitinib and deuterated ruxolitinib analogs, there is a continuing need for improved methods for synthesizing ruxolitinib and deuterated forms thereof.

### SUMMARY OF THE INVENTION

The present invention provides improved compounds and methods for synthesizing intermediates useful for preparing ruxolitinib, deuterated forms of ruxolitinib, and other JAK inhibitors. In one aspect, the invention provides a process for preparing a compound of Formula 5:

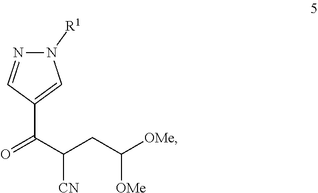

the process comprising the step of reacting a compound of Formula 1:

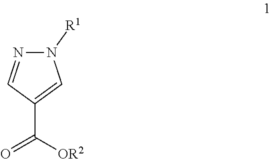

with Compound 4

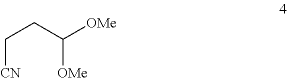

and a base (e.g., a base selected from lithium hexamethyldisilazide (LiHMDS) and sodium hexamethyldisilazide (NaHMDS)); wherein R¹ is selected from H or a protecting group (PG), and wherein R² is C₁-C₄ alkyl.

In another aspect, the invention provides a process for preparing a compound of Formula 7:

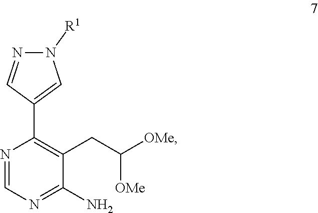

the process comprising the step of reacting a compound of Formula 5:

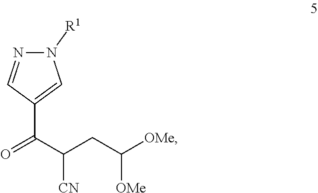

with formamidine or a salt thereof,
wherein R¹ is selected from H or a protecting group (PG).

In another aspect, the invention provides a process for preparing a compound of Formula 7, the process comprising the step of reacting a compound of Formula 5 with formamidine or a salt thereof; or with an ammonium source and trialkyl orthoformate; wherein R¹ is selected from H or a protecting group (PG).

In another aspect, the invention provides a process for preparing a compound of Formula 6a:

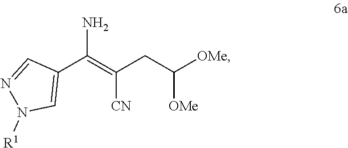

the process comprising the step of reacting a compound of Formula 5:

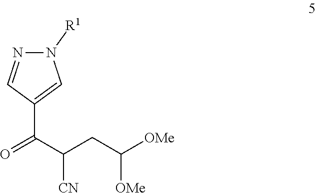

with an ammonium salt;
wherein R¹ is selected from H or a protecting group (PG).

In another aspect, the invention provides a process for preparing a compound of Formula 6a, the process comprising the step of reacting a compound of Formula 5 with an ammonium source such as an ammonium salt or ammonia;
wherein R¹ is selected from H or a protecting group (PG). In certain embodiments, the ammonium source is an ammonium salt. In certain embodiments, the ammonium salt is ammonium formate, ammonium chloride or ammonium acetate.

In another aspect, the invention provides a process for preparing a compound of Formula 7:

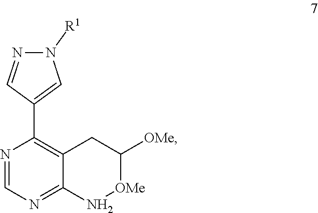

the process comprising the step of reacting a compound of Formula 6a:

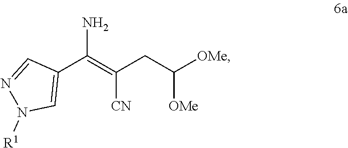

with formamidine or a salt thereof;
wherein R¹ is selected from H or a protecting group (PG).

Other aspects and embodiments of the invention will be appreciated from the detailed description and claims herein.

### DETAILED DESCRIPTION OF THE INVENTION

### Definitions

The term “alkyl” refers to a monovalent saturated hydrocarbon group. C₁-C₆ alkyl is an alkyl having from 1 to 6 carbon atoms; C₁-C₄ alkyl is an alkyl having from 1 to 4 carbon atoms. In some embodiments, an alkyl may be linear or branched. In some embodiments, an alkyl may be primary, secondary, or tertiary. Non-limiting examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl. Non-limiting examples of primary alkyl groups include methyl, ethyl, n-propyl, n-butyl, n-pentyl, and n-hexyl. Non-limiting examples of secondary alkyl groups include isopropyl, sec-butyl, and 2-methylpentyl. Non-limiting examples of tertiary alkyl groups include t-butyl.

The term “alkenyl” refers to a monovalent unsaturated hydrocarbon group where the unsaturation is represented by a double bond. C₂-C₆ alkenyl is an alkenyl having from 2 to 6 carbon atoms. An alkenyl may be linear or branched. Examples of alkenyl groups include CH₂═CH— (vinyl), CH₂═C(CH₃)—, CH₂═CH—CH₂— (allyl), CH₃—CH═CH—CH₂-(crotyl), CH₃—CH═C(CH₃)— and CH₃—CH═CH—CH(CH₃)—CH₂—. Where double bond stereoisomerism is possible, the stereochemistry of an alkenyl may be (E), (Z), or a mixture thereof.

“Aryl” by itself or as part of another substituent refers to a monocyclic or polycyclic monovalent aromatic hydrocarbon group having the stated number of carbon atoms (i.e., C₅-C₁₄ means from 5 to 14 carbon atoms). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octophene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthylene, and the like. In a specific embodiment, the aryl group is cyclopentadienyl, phenyl or naphthyl. In a more specific embodiment, the aryl group is phenyl or naphthyl.

The term “heterocyclic” refers to a monocyclic or bicyclic monovalent saturated or non-aromatic unsaturated ring system wherein from 1 to 4 ring atoms are heteroatoms independently selected from the group consisting of O, N and S. The term “3 to 10-membered heterocycloalkyl” refers to a heterocycloalkyl wherein the number of ring atoms is from 3 to 10. Examples of 3 to 10-membered heterocycloalkyl include 3 to 6-membered heterocycloalkyl. Bicyclic ring systems include fused, bridged, and spirocyclic ring systems. More particular examples of heterocycloalkyl groups include azepanyl, azetidinyl, aziridinyl, imidazolidinyl, morpholinyl, oxazolidinyl, oxazolidinyl, piperazinyl, piperidinyl, pyrazolidinyl, pyrrolidinyl, quinuclidinyl, and thiomorpholinyl.

In the above heterocyclic substituents, the nitrogen, phosphorus, carbon or sulfur atoms can be optionally oxidized to various oxidation states. In a specific example, the group —S(0)₀₋₂—, refers to —S—(sulfide), —S(O)—(sulfoxide), and —SO₂— (sulfone) respectively. For convenience, nitrogens, particularly but not exclusively, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms can be optionally quaternized; and the ring substituent can be partially or fully saturated or aromatic.

“CTP-543” is a deuterated analog of ruxolitinib, known by the chemical name (R)-3-(4-(7H-pyrrolo[2,3-d]pyrimidin-4-yl)-1H-pyrazol-1-yl)-3-(cyclopentyl-2,2,3,3,4,4,5,5-ds)propanenitrile. Compound (I) may also be referred to herein as Ds-ruxolitinib. Compound (I) is represented by the following structural formula:

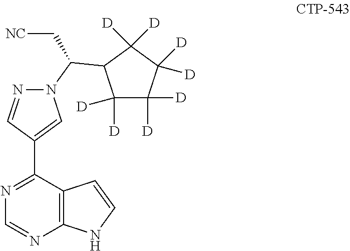 [SMILES: [2H]C1([2H])C([C@@H](CC#N)n2cc(-c3ncnc4[nH]ccc34)cn2)C([2H])([2H])C([2H])([2H])C1([2H])[2H]]

As used herein, the terms “contacting” and “reacting” are used as known in the art and generally refer to the bringing together of chemical reagents in such a manner so as to allow their interaction at the molecular level to achieve a chemical or physical transformation. In some embodiments, contacting or reacting involves two (or more) reagents, wherein one or more equivalents of a second reagent are used with respect to a first reagent. The reacting steps of the processes described herein can be conducted for a time and under conditions suitable for preparing the identified product.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Thus, for example, a nitrogen atom can be protected as a carbamate, e.g., with a protecting group such as t-butoxycarbonyl (Boc); as a sulfonamide, e.g., with a protecting group such as triflyl (Tf, SO₂—CF₃); as an amide, e.g., with a protecting group such as acetyl, benzoyl, or trifluoroacetyl (F₃—Ac); as an amine, e.g., with a protecting group such as benzyl or trityl (Tr, —CPh₃); or as a silyl amine (e.g., with a protecting group such as SiPh₂Bu^{t}). Adjustments to the protecting groups and formation and cleavage methods described herein may be adjusted as necessary in light of the various substituents.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane (DCM), tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, trifluorotoluene (TFT), and mixtures thereof.

Suitable ether solvents include: dimethoxymethane, tetrahydrofuran (THF), 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof. Additional ether solvents include 2-methyltetrahydrofuran and cyclopentyl methyl ether (and mixtures thereof, including with other ether solvents described herein).

Suitable protic solvents can include, by way of example and without limitation, water, methanol (MeOH), ethanol (EtOH), isopropanol (iPrOH), 2-nitroethanol, 2-fluoroethanol, 2,2,2-trifluoroethanol (TFE), ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, hexafluoroisopropanol (HFIP), acetic acid (AcOH), and mixtures thereof.

Suitable aprotic solvents can include, by way of example and without limitation, tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide (DMSO), propionitrile, ethyl formate, methyl acetate, hexachloroacetone, acetone, ethyl methyl ketone, ethyl acetate (EtOAc), sulfolane, N,N-dimethylpropionamide, tetramethylurea, nitromethane, nitrobenzene, hexamethylphosphoramide, and mixtures thereof.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, naphthalene, and mixtures thereof.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures). “Elevated temperature” refers to temperatures above room temperature (about 22° C.).

The reactions of the processes described herein can be carried out in air or under an inert atmosphere. Typically, reactions containing reagents or products that are substantially reactive with air can be carried out using air-sensitive synthetic techniques that are well known to the skilled artisan.

Examples of acids can be inorganic or organic acids. Non-limiting examples of inorganic acids include hydrochloric acid, hydrobromic acid, sulfuric acid, phosphoric acid, and nitric acid. Non-limiting examples of organic acids include formic acid, acetic acid, propionic acid, butanoic acid, benzoic acid, 4-nitrobenzoic acid, methanesulfonic acid, p-toluenesulfonic acid, benzenesulfonic acid, tartaric acid, trifluoroacetic acid, propiolic acid, butyric acid, 2-butynoic acid, vinyl acetic acid, pentanoic acid, hexanoic acid, heptanoic acid, octanoic acid, nonanoic acid and decanoic acid.

Non-limiting examples of bases include lithium hydroxide, sodium hydroxide, potassium hydroxide, lithium carbonate, sodium carbonate, and potassium carbonate. Some example strong bases include, but are not limited to, hydroxide, alkoxides, metal amides, metal hydrides, metal dialkylamides and metal silylamides (including, e.g., lithium hexamethyldisilazide (LiHMDS) and sodium hexamethyldisilazide (NaHMDS)) and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium and potassium salts of methyl, ethyl, n-propyl, i-propyl, n-butyl, t-butyl, trimethylsilyl and cyclohexyl substituted amides.

Upon carrying out preparation of compounds according to the processes described herein, the usual isolation and purification operations such as concentration, filtration, extraction, solid-phase extraction, recrystallization, chromatography, and the like may be used to isolate the desired products.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes salt forms of the compounds described herein. A salt of a compound of this invention is formed between an acid and a basic group of the compound, such as an amino functional group, or a base and an acidic group of the compound, such as a carboxyl functional group. According to one embodiment, the compound is a pharmaceutically acceptable acid addition salt. In one embodiment the acid addition salt may be a deuterated acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In one embodiment, the acids commonly employed to form pharmaceutically acceptable salts include the above-listed inorganic acids, wherein at least one hydrogen is replaced with deuterium.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “Sec” or “s-” each refer to secondary. “n-” refers to normal. “i-” refers to iso. “US” refers to the United States of America. Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Processes

In one aspect, the invention provides a process for preparing a compound of Formula A:

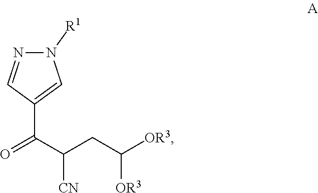

the process comprising the step of reacting a compound of Formula 1:

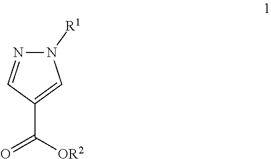

with a compound of Formula D:

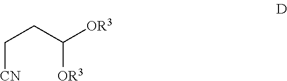

and a base (e.g., a base selected from lithium hexamethyldisilazide (LiHMDS) and sodium hexamethyldisilazide (NaHMDS)); wherein R¹ is selected from H and a protecting group (PG), wherein R² is selected from C₁-C₁₀ alkyl (e.g., methyl or ethyl), C₂-C₁₀ alkenyl (e.g., allyl), aryl, and heterocyclic, and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl (—CH₂-phenyl). In certain embodiments, R² is methyl. In certain embodiments, R² is ethyl. In certain embodiments, R³ is methyl. In certain embodiments, R³ is ethyl. In certain embodiments, the step of reacting is performed in an aprotic solvent such as tetrahydrofuran (THF). In certain embodiments, the step of reacting is performed under an inert atmosphere (e.g., a nitrogen atmosphere). In certain embodiments, the step of reacting is performed at a temperature between −20° C. and 20° C., e.g., between −20° C. and 10° C., between −15° C. and 0° C., or between −10° C. and 0° C.

In another aspect, the invention provides a process for preparing a compound of Formula E:

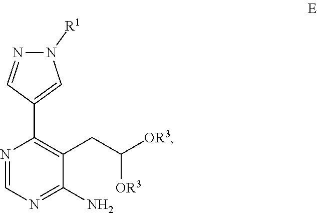

the process comprising the step of reacting a compound of Formula A:

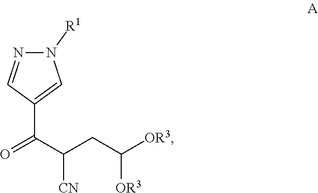

with formamidine or a salt thereof,

wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, each R³ is ethyl and R¹ is a protecting group. In certain embodiments, if each R³ is ethyl, R¹ is not H. In certain embodiments, the step of reacting is performed in an aprotic solvent such as bis(2-methyoxyethyl)ether (diglyme). In certain embodiments, the step of reacting is performed in protic solvent such as methanol. In certain embodiments, the step of reacting is performed under an inert atmosphere (e.g., a nitrogen atmosphere). In certain embodiments, the step of reacting is performed at a temperature between 20° C. and 180° C., e.g., between 50° C. and 165° C. In certain embodiments, the formamidine is formamidine acetate. In another aspect, the invention provides a process for preparing a compound of Formula E, the process comprising the step of reacting a compound of Formula A with formamidine or a salt thereof, or with an ammonium source and trialkyl orthoformate; or with an ammonium source and dimethylformamide dimethyl acetal; wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, R³ is ethyl. In certain embodiments, R³ is ethyl and R¹ is a protecting group. In certain embodiments, if R³ is ethyl, R¹ is not H. In certain embodiments, the step of reacting is performed in an aprotic solvent such as bis(2-methyoxyethyl)ether (diglyme). In certain embodiments, the step of reacting is performed in protic solvent such as methanol. In certain embodiments, the step of reacting is performed under an inert atmosphere (e.g., a nitrogen atmosphere). In certain embodiments, the step of reacting is performed at a temperature between 20° C. and 180° C., e.g., between 50° C. and 165° C. In certain embodiments, the process comprising the step of reacting a compound of Formula A with an ammonium source and trialkyl orthoformate. In certain embodiments, the trialkyl orthoformate is trimethyl orthoformate. In certain embodiments, the ammonium source is ammonia. In certain embodiments, the ammonium source is an ammonium salt. In certain embodiments, the ammonium salt is ammonium acetate. In certain embodiments, the trialkyl orthoformate is selected from trimethyl orthoformate and triethyl orthoformate. In certain embodiments, the process comprises the step of reacting a compound of Formula A with ammonium acetate and trimethylorthoformate.

In another aspect, the invention provides a process for preparing a compound of Formula B:

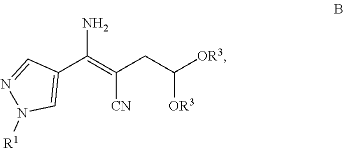

a compound of Formula C:

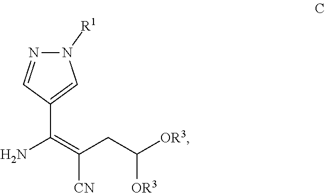

or a mixture thereof, the process comprising the step of reacting a compound of Formula A:

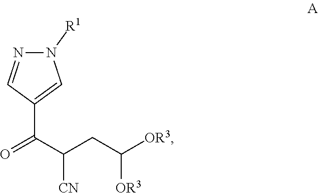

with an ammonium salt; wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, R³ is ethyl. In certain embodiments, the step of reacting is performed in a protic solvent such as ethanol, e.g., anhydrous ethanol, an aprotic solvent such as bis(2-methyoxyethyl)ether (diglyme). In certain embodiments, the step of reacting is performed in a protic solvent such as methanol, ethanol, or n-butanol, e.g., anhydrous methanol, ethanol, or n-butanol. In certain embodiments, the step of reacting is performed under an inert atmosphere (e.g., a nitrogen atmosphere). In certain embodiments, the step of reacting is performed at a temperature between 20° C. and 120° C., e.g., between 20° C. and 100° C. In certain embodiments, the ammonium salt is ammonium formate. In certain embodiments, the process produces a compound of Formula B. In certain embodiments, the process produces a compound of Formula C. In certain embodiments, the process produces a mixture of a compound of Formula B and a compound of Formula C.

In another aspect, the invention provides a process for preparing a compound of Formula B, a compound of Formula C, or a mixture thereof the process comprising the step of reacting a compound of Formula A, with an ammonia source such as ammonia or an ammonium salt; wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, R³ is ethyl. In certain embodiments, the step of reacting is performed in a protic solvent such as methanol, ethanol, n-butanol, e.g., anhydrous methanol, ethanol, or n-butanol, or an aprotic solvent such as bis(2-methyoxyethyl)ether (diglyme). In certain embodiments, the step of reacting is performed under an inert atmosphere (e.g., a nitrogen atmosphere). In certain embodiments, the step of reacting is performed at a temperature between 20° C. and 120° C., e.g., between 20° C. and 100° C. In certain embodiments, the process comprises the step of reacting a compound of Formula A with ammonium formate. In certain embodiments, the process comprises the step of reacting a compound of Formula A with ammonium acetate. In certain embodiments, the process comprises the step of reacting a compound of Formula A with ammonia. In certain embodiments, the process produces a compound of Formula B. In certain embodiments, the process produces a compound of Formula C. In certain embodiments, the process produces a mixture of a compound of Formula B and a compound of Formula C.

In another aspect, the invention provides a process for preparing a compound of Formula E:

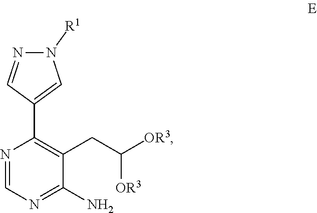

the process comprising the step of reacting a compound of Formula B:

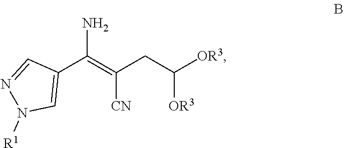

a compound of Formula C:

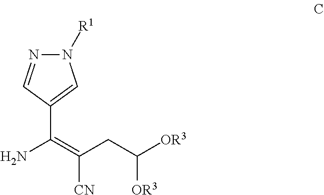

C or a mixture thereof
with formamidine or a salt thereof,
wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, R³ is ethyl. In certain embodiments, R³ is ethyl and R¹ is a protecting group. In certain embodiments, R³ is ethyl and R¹ is not H. In certain embodiments, the step of reacting is performed in a protic solvent such as n-butanol. In certain embodiments, the step of reacting is performed in a protic solvent such as methanol, NH₃/methanol or n-butanol. In certain embodiments, the step of reacting is performed in an aprotic solvent such as toluene. In certain embodiments, the step of reacting is performed under an inert atmosphere (e.g., a nitrogen atmosphere). In certain embodiments, the step of reacting is performed at a temperature between 20° C. and 150° C., e.g., between 50° C. and 140° C. In certain embodiments, the formamidine is formamidine acetate. In certain embodiments, the process comprises the step of reacting a compound of Formula B with formamidine or a salt thereof. In certain embodiments, the process comprises the step of reacting a compound of Formula C with formamidine or a salt thereof. In certain embodiments, the process comprises the step of reacting a mixture of a compound of Formula B and a compound of Formula C with formamidine or a salt thereof.

In another aspect, the invention provides a process for preparing a compound of Formula E, the process comprising the step of reacting a compound of Formula B, or a compound of Formula C with formamidine or a salt thereof, or with trialkyl orthoformate (such as trimethyl orthoformate or triethyl orthoformate) and an ammonium source, or with dimethylformamide dimethyl acetal and an ammonium source; wherein R¹ is selected from H and a protecting group (PG), and wherein each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is H. In certain embodiments, R¹ is a protecting group. In certain embodiments, R¹ is a protecting group which is benzyl. In certain embodiments, R³ is methyl. In certain embodiments, R³ is ethyl. In certain embodiments, R³ is ethyl and R¹ is a protecting group. In certain embodiments, R³ is ethyl and R¹ is not H. In certain embodiments, the step of reacting is performed in a protic solvent such as methanol, NH₃/methanol or n-butanol. In certain embodiments, the step of reacting is performed in an aprotic solvent such as toluene. In certain embodiments, the step of reacting is performed under an inert atmosphere (e.g., a nitrogen atmosphere). In certain embodiments, the step of reacting is performed at a temperature between 20° C. and 150° C., e.g., between 50° C. and 140° C. In certain embodiments, the process comprises the step of reacting a compound of Formula B with trimethyl orthoformate. In certain embodiments, the process comprises the step of reacting a compound of Formula C with trimethyl orthoformate. In certain embodiments, the process comprises the step of reacting a compound of Formula B and a compound of Formula C with trimethyl orthoformate. In certain embodiments, the process comprises the step of reacting a compound of Formula B with dimethylformamide dimethyl acetal. In certain embodiments, the process comprises the step of reacting a compound of Formula C with dimethylformamide dimethyl acetal. In certain embodiments, the process comprises the step of reacting a compound of Formula B and a compound of Formula C with dimethylformamide dimethyl acetal.

In certain embodiments, the ammonium source is ammonia or an ammonium salt. In certain embodiments, the ammonium salt is ammonium formate, ammonium chloride or ammonium acetate.

In one aspect, the invention provides processes for preparing a compound of Formula 7, an intermediate useful for synthesizing ruxolitinib, CTP-543, and other JAK inhibitors. In certain embodiments, the methods comprise the steps shown in Scheme 1 below:

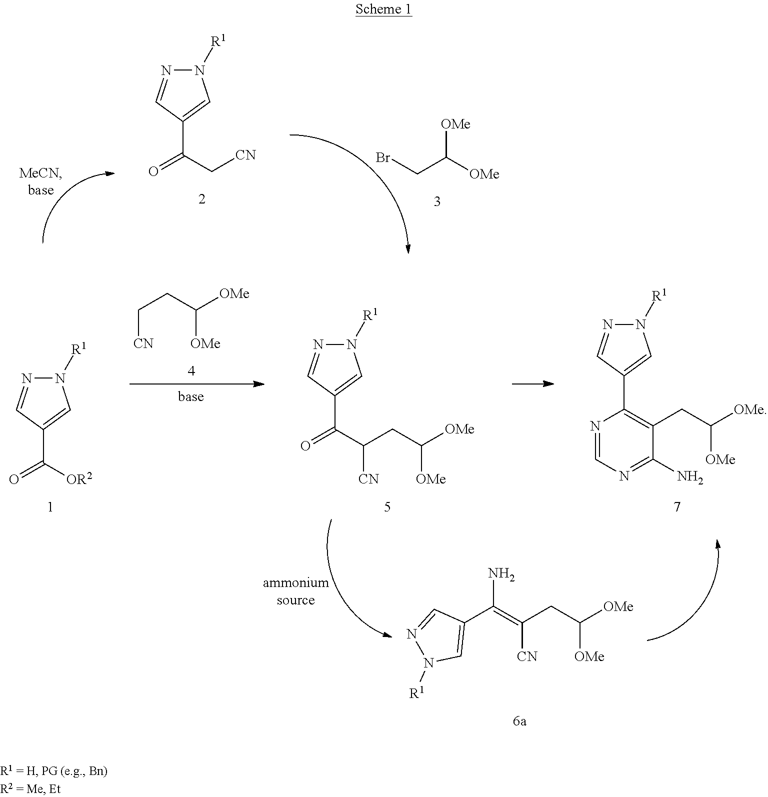

In certain embodiments, the process for preparing a compound of Formula 7 comprises the step of reacting a compound of Formula 6a with formamidine or a salt thereof, or with trimethyl orthoformate, or with dimethylformamide dimethyl acetal.
In other embodiments, the methods comprise the steps shown in Scheme 2 below:

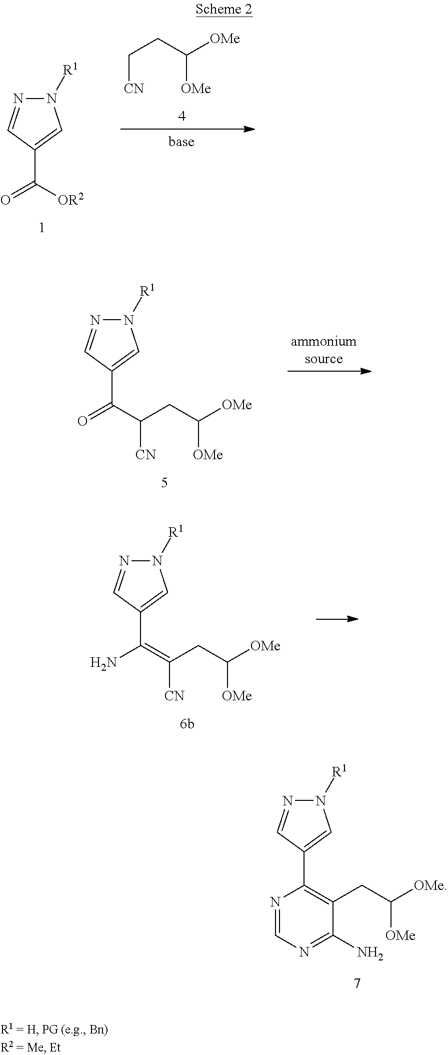

In certain embodiments, the process for preparing a compound of Formula 7 comprises the step of reacting a compound of Formula 6b with formamidine or a salt thereof; or with trimethyl orthoformate, or with dimethylformamide dimethyl acetal.
In certain embodiments, the methods comprise the steps shown in Scheme 3 below:

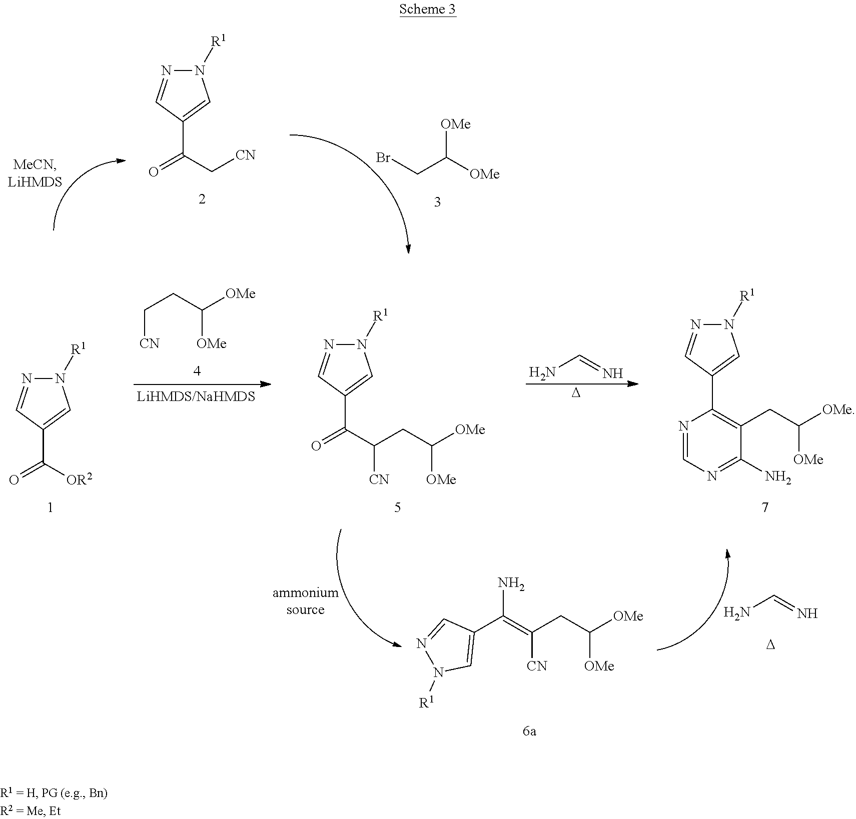

In one aspect, Compound 8, i.e., a compound of Formula 7 in which R¹ is H, may be used as an intermediate in a process for preparing ruxolitinib, e.g., as shown in Scheme 4 below:

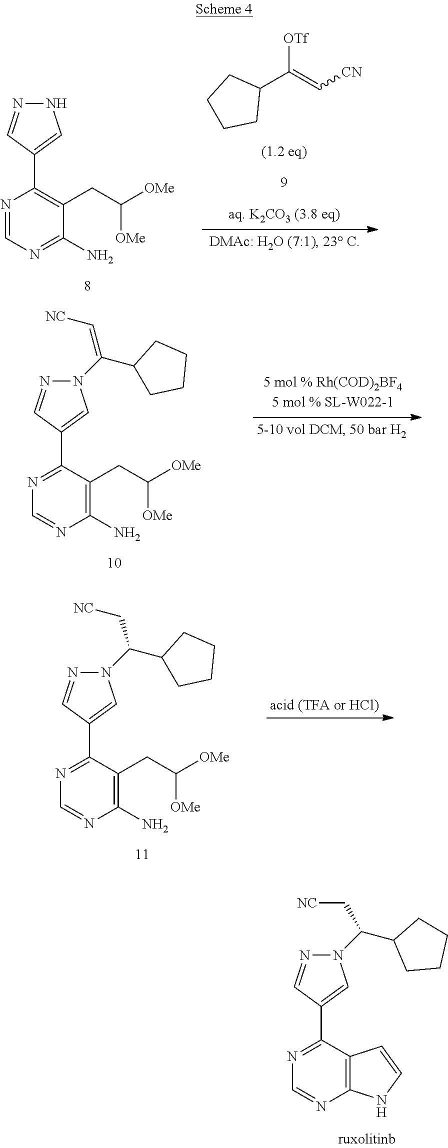 [SMILES: COC(Cc1c(N)ncnc1-c1cn[nH]c1)OC ; N#CC=C(OS(=O)(=O)C(F)(F)F)C1CCCC1 ; COC(Cc1c(N)ncnc1-c1cnn(/C(=C\C#N)C2CCCC2)c1)OC ; COC(Cc1c(N)ncnc1-c1cnn([C@H](CC#N)C2CCCC2)c1)OC ; N#CC[C@H](C1CCCC1)n1cc(-c2ncnc3[nH]ccc23)cn1]

Treatment of ruxolitinib produced by the above process with phosphoric acid (H₃PO₄) produces the phosphate salt of ruxolitinib.

In another aspect, Compound 8, i.e., a compound of Formula 7 in which R¹ is H, may be used as an intermediate in a process for preparing CTP-543, as shown in Scheme 5 below:

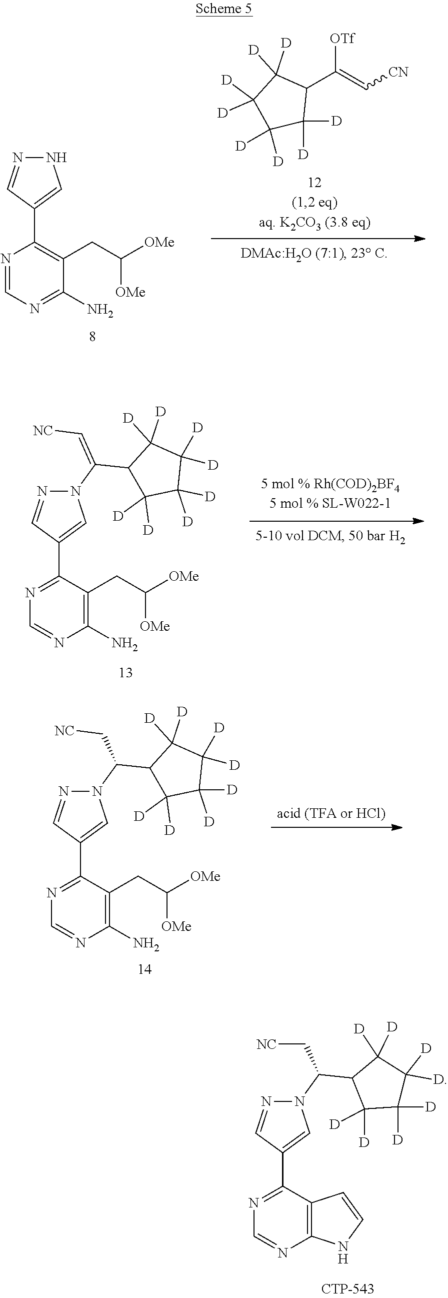

Treatment of CTP-543 produced by the above process with phosphoric acid (H₃PO₄) produces the phosphate salt of CTP-543.

### Intermediates

In one aspect, the invention provides compounds and intermediates useful for preparing ruxolitinib, deuterated analogs of ruxolitinib, and other JAK inhibitors. See, e.g., PCT Publication WO2020/163653.

In certain embodiments, the invention provides a compound represented by the structure:

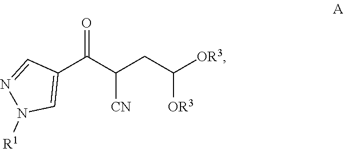

or a salt thereof, in which R¹ is H or a protecting group, and each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, if each R³ is ethyl, R¹ is not H. In certain embodiments, R¹ is a benzyl group. In certain embodiments, R¹ is H. In certain embodiments, each R³ is methyl.

In one embodiment, the invention provides a compound represented by the structure:

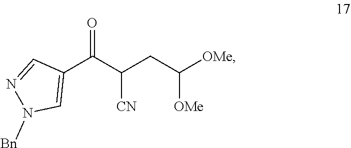 [SMILES: COC(CC(C#N)C(=O)c1cnn(Cc2ccccc2)c1)OC]

or a salt thereof.

In another embodiment, the invention provides a compound represented by the structure:

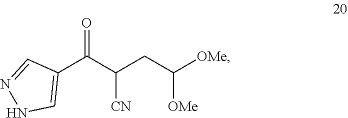 [SMILES: COC(CC(C#N)C(=O)c1cn[nH]c1)OC]

or a salt thereof.

In certain embodiments, the invention provides a compound represented by the structure:

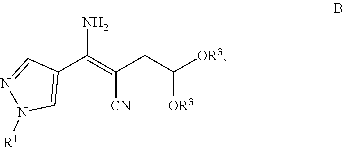

or a salt thereof, in which R¹ is H or a protecting group, and each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is a benzyl group. In certain embodiments, R¹ is H. In certain embodiments, each R³ is methyl. In certain embodiments, each R³ is ethyl. In certain embodiments, if each R³ is ethyl, R¹ is not H.

In another embodiment, the invention provides a compound represented by the structure:

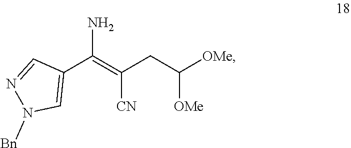 [SMILES: COC(C/C(C#N)=C(\N)c1cnn(Cc2ccccc2)c1)OC]

or a salt thereof.

In another embodiment, the invention provides a compound represented by the structure:

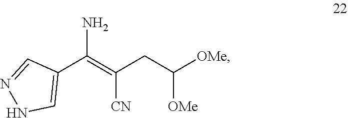 [SMILES: COC(C/C(C#N)=C(\N)c1cn[nH]c1)OC]

or a salt thereof.

In certain embodiments, the invention provides a compound represented by the structure:

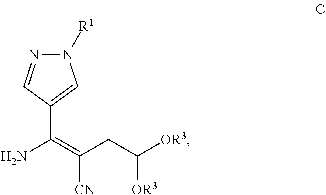

or a salt thereof, in which R¹ is H or a protecting group, and each R³ is C₁-C₁₀ alkyl (e.g., methyl or ethyl), C₂-C₁₀ alkenyl (e.g., allyl), aryl, or the two R³'s, taken together with the oxygen atoms to which they are attached, form a 5-7-membered heterocyclic ring which may optionally be substituted (e.g., a 1,3-dioxolan-2-yl ring, or a 1,3-dioxan-2-yl ring, or a 1,3-benzodioxolan-2-yl ring, each optionally substituted with one or more methyl groups). In certain embodiments, R¹ is a benzyl group. In certain embodiments, R¹ is H. In certain embodiments, each R³ is methyl. In certain embodiments, each R³ is ethyl. In certain embodiments, if each R³ is ethyl, R¹ is not H.

In another embodiment, the invention provides a compound represented by the

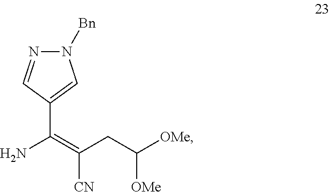 [SMILES: COC(C/C(C#N)=C(/N)c1cnn(Cc2ccccc2)c1)OC]

or a salt thereof.

In another embodiment, the invention provides a compound represented by the structure:

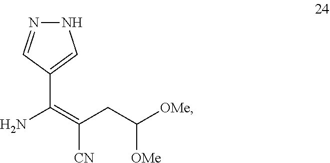 [SMILES: COC(C/C(C#N)=C(/N)c1cn[nH]c1)OC]

or a salt thereof.

### Examples

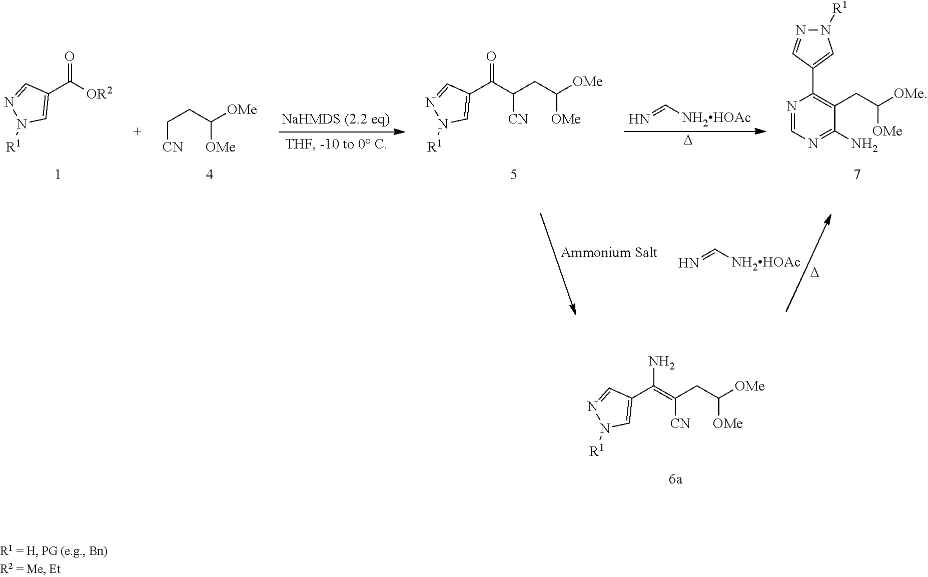

### Example 1: Preparation of Methyl 1-Benzyl-1H-pyrazole-4-carboxylate (16)

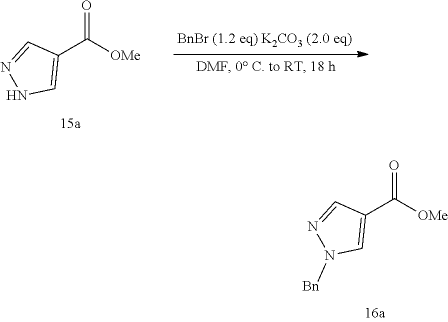 [SMILES: COC(=O)c1cn[nH]c1 ; COC(=O)c1cnn(Cc2ccccc2)c1]

To a 250 ml jacketed flask with stir-bar, thermocouple and positive nitrogen stream, were added methyl 1H-pyrazole-4-carboxylate 15a (10 g, 77.7 mmol, 1.0 equiv.) and K₂CO₃ (21.9 g, 158 mmol, 2.04 equiv.) followed by dimethylformamide (DMF) (80 ml). The mixture was cooled to 0° C. Benzyl bromide (11.3 ml, 93.3 mmol, 1.2 equiv.) was charged over 10 minutes. The reaction mixture was brought to room temperature and stirred for 18 hours. Water (50 ml) was added and the mixture was transferred to a 500 ml separating funnel. The mixture was extracted twice with ethyl acetate (150 ml). The combined organic extract was washed with brine (30 ml), dried over Na₂SO₄, and concentrated in vacuo to give a colorless residue, which crystallized upon standing to provide methyl 1-benzyl-1H-pyrazole-4-carboxylate 16a (14.1 g, 85% yield).

¹H-NMR (400 MHz, CDCl₃): δ 7.85 (s, 1H), 7.75 (s, 1H), 7.31-7.22 (m, 3H), 7.18-7.13 (m, 2H), 5.21 (s, 2H), 3.71 (s, 3H).

### Example 2: Preparation of 2-(1-Benzyl-1H-pyrazole-4-carbonyl)-4,4-dimethoxybutanenitrile (17)

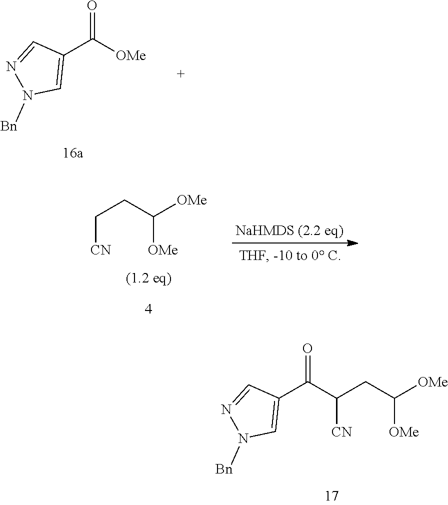 [SMILES: COC(=O)c1cnn(Cc2ccccc2)c1 ; COC(CCC#N)OC ; COC(CC(C#N)C(=O)c1cnn(Cc2ccccc2)c1)OC]

To a 125 ml jacketed flask with thermocouple, stir-bar and positive stream of nitrogen was added sodium bis(trimethylsilyl)amide (44 ml, 87.5 mmol, 2.2 equiv, 2 M in tetrahydrofuran (THF)) and 10 ml anhydrous THF. The mixture was cooled to −14° C. A solution of compound 1-benzyl-1H-pyrazole-4-carboxylate 16a (8.6 g, 39.8 mmol, 1.0 equiv.) and 4,4-dimethoxybutanenitrile 4 (6.2 ml, 47.7 mmol, 1.2 equiv.) in 15 ml THF was charged to the mixture over 40 minutes. After addition, the mixture was stirred at −10° C. for 1 hours, then stirred at 0° C. overnight. The reaction mixture was acidified using hydrochloric acid (0.5 N) to pH=2 then extracted twice with ethyl acetate (150 ml). The combined organic extracts were washed with water (20 ml), brine (20 ml), then dried over Na₂SO₄ and concentrated in vacuo to give a colorless oil residue. The residue was purified by flash chromatography with ethyl acetate/heptane (1:1) to give product 2-(1-benzyl-1H-pyrazole-4-carbonyl)-4,4-dimethoxybutanenitrile 17 (10.9 g, 87% yield) as a colorless oil.

¹H-NMR (400 MHz, CDCl₃): δ 8.05 (s, 1H), 7.99 (s, 1H), 7.42-7.34 (m, 3H), 7.30-7.25 (m, 2H), 5.37-5.28 (m, 2H), 4.52 (dd, 1H), 4.05 (m, 1H), 3.39 (s, 3H), 3.30 (s, 3H), 2.30 (m, 1H), 2.18 (m, 1H).

### Example 3: Preparation of 2-(Amino(1-benzyl-1H-pyrazol-4-yl)methylene)-4,4-dimethoxybutanenitrile (18)

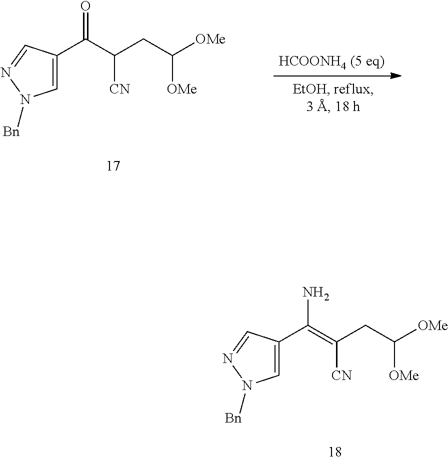 [SMILES: COC(CC(C#N)C(=O)c1cnn(Cc2ccccc2)c1)OC ; COC(C/C(C#N)=C(\N)c1cnn(Cc2ccccc2)c1)OC]

To a 125 ml jacketed flask with stir-bar, thermocouple and positive nitrogen stream, was added compound 2-(1-benzyl-1H-pyrazole-4-carbonyl)-4,4-dimethoxybutanenitrile 17 (2.66 g, 8.49 mmol, 1.0 equiv.) and ammonium formate (3.0 g, 42.4 mmol, 5 equiv.) followed by anhydrous ethanol (25 ml) and 0.6 g of 3 Å molecular sieves. The mixture was heated at reflux for 18 hours. The reaction mixture was filtered through a short silica plug, followed by an ethanol wash (5 mL). The resulting filtrate was concentrated in vacuo to give pale brown residue, which was purified by column chromatography using dichloromethane/methanol (10:1) to provide 2-(amino(1-benzyl-1H-pyrazol-4-yl)methylene)-4,4-dimethoxybutanenitrile 18 (1.82 g, 69% yield, E:Z=9:1) as yellow oil. The E-geometry of the major enamine isomer was confirmed by NOESY data.

¹H-NMR (400 MHz, CDCl₃): δ 7.99 (s, 1H), 7.80 (s, 1H), 7.38-7.30 (m, 3H), 7.27-7.22 (m, 2H), 5.31 (s, 2H), 4.83 (bs, 2H), 4.45 (t, 1H), 3.43 (s, 6H), 2.51 (d, 2H).

### Example 4: Preparation of 6-(1-Benzyl-1H-pyrazol-4-yl)-5-(2,2-dimethoxyethyl)pyrimidin-4-amine (19)

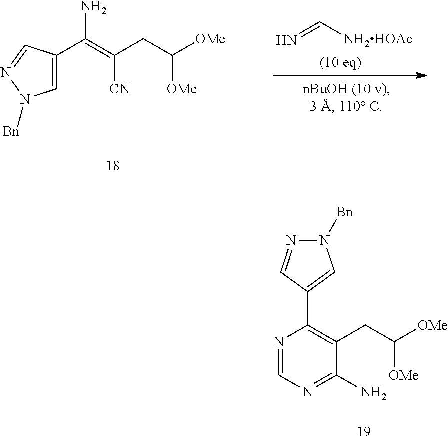

To a 25 ml two neck flask with condenser, stir-bar, thermocouple and positive nitrogen stream, was added compound (E)-2-(amino(1-benzyl-1H-pyrazol-4-yl)methylene)-4,4-dimethoxybutanenitrile 18 (0.40 g, 1.28 mmol, 1.0 equiv.) and formamidine acetate (0.40 g, 3.84 mmol, 3 equiv.) followed by n-butanol (8 ml) and 100 mg of 3 Å molecular sieves. The resulting mixture was heated to reflux for 18 hours. Additional formamidine acetate (0.27 g, 2.0 equiv.) was charged and reflux was continued for 36 hours. Additional formamidine acetate (810 mg, 5.0 equiv.) was added over 3 days while maintaining reflux temperature. After a total of 6 days, an aliquot of reaction mixture analysis indicated >80% conversion to 6-(1-benzyl-1H-pyrazol-4-yl)-5-(2,2-dimethoxyethyl)pyrimidin-4-amine 19, which was identified by HPLC-MS by comparison to a reference marker.

UV max: 240 and 290; LCMS (ESI, positive mode): Expected: 340.2 (M+H); Found: 340.1 (M+H).

### Example 5a: Preparation of 4,4-Dimethoxy-2-(1H-pyrazole-4-carbonyl)butanenitrile (20)

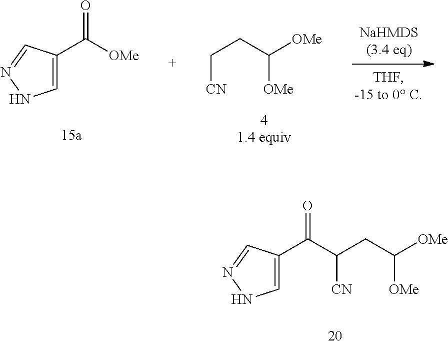 [SMILES: COC(=O)c1cn[nH]c1 ; COC(CCC#N)OC ; COC(CC(C#N)C(=O)c1cn[nH]c1)OC]

To a 250 ml jacketed flask with thermocouple, stir-bar and slow stream of nitrogen, was added sodium bis(trimethylsilyl)amide (NaHMDS) (67.5 ml, 135 mmol, 3.4 equiv, 2 M in THF) and it was cooled to −14° C. A solution of methyl pyrazole-4-carboxylate 15a (5 g, 39.6 mmol, 1.0 equiv.) and 4,4-dimethoxy butanenitrile 4 (7.3 ml, 55.5 mmol, 1.4 equiv.) in 15 ml THF was added to the solution of sodium bis(trimethylsilyl)amide over 3 hours. The mixture was stirred at −10° C. for 1 hour, then stirred overnight at 0° C. The reaction mixture was cooled to −10° C. and acidified to pH=2 using hydrochloric acid (0.5 N). The solution was then transferred to 500 ml separating funnel and extracted twice with ethyl acetate (100 ml). The combined organic layers were washed with water (20 ml), brine (20 ml), dried over Na₂SO₄, and concentrated in vacuo to provide a colorless oil. The residue was purified by flash chromatography with ethyl acetate/heptane (8:2) to provide 4,4-dimethoxy-2-(1H-pyrazole-4-carbonyl)butanenitrile 20 (6.0 g, 61% yield) as a colorless oil.

¹H-NMR (400 MHz, CDCl₃): δ 8.24 (s, 2H), 4.55 (dd, 1H), 4.17 (dd, 1H), 3.41 (s, 3H), 3.34 (s, 3H), 2.35 (m, 1H), 2.24 (m, 1H).

### Example 5b: Preparation of 4,4-Dimethoxy-2-(1H-pyrazole-4-carbonyl)butanenitrile (20)

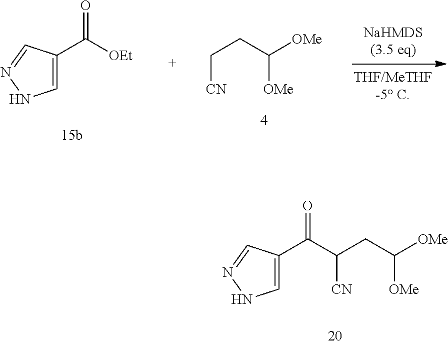 [SMILES: CCOC(=O)c1cn[nH]c1 ; COC(CCC#N)OC ; COC(CC(C#N)C(=O)c1cn[nH]c1)OC]

To a 2 M solution of NaHMDS in THF (375 mL, 749 mmol, 3.5 equiv.) was added THF (53.5 mL, 1.8 vol) and the solution was cooled to −5° C. A solution of ethyl 4-pyrazolecarboxylate 15b (30.0 g, 214 mmol, 1.0 equiv.) in THF (33 mL, 1.1 vol) was then added rinsing in with additional THF (5.0 mL, 0.2 vol). To the resulting orange suspension was added a solution of 3-cyanopropionaldehyde dimethyl acetal 4 (36.0 g, 278 mmol, 1.3 equiv.) in THF (72 mL, 2.4 vol) over a period of 6 hours at a temperature of −5 to 0° C. The reaction mixture was then held at this temperature for an additional 15 hours then water (180 mL, 6.0 vol) was added while maintaining the temperature ≤5° C. The agitation was stopped and the upper organic layer removed. The aqueous phase was adjusted to pH ˜11 with 6N HCl (92 mL, 3.0 vol) then washed with 2-MeTHF (2×120 mL, 2×4 vol). The organic phases were discarded and n-butanol (150 mL. 5 vol) was added to the remaining aqueous solution. The resulting mixture was adjusted to pH 5 with 85% phosphoric acid (˜8 mL) then the stirring was stopped and the layers were separated. The organic layer was collected and the remaining aqueous solution was further extracted with n-butanol (150 mL, 5 vol). The organic layers were combined, washed with water (100 mL, 3.3 vol) then concentrated in vacuo to a target volume of 120 mL (4 vol) to afford dimethyl acetal 20 as a red/orange clear solution in n-butanol (135.6 g, 28.2% w/w by QNMR (Quantitative ¹H-NMR) assay: 38.2 g of 20, 80% yield).

### Example 6a: Preparation of 5-(2,2-Dimethoxvethyl)-6-(1H-pyrazol-4-yl)pyrimidin-4-amine (8) 8

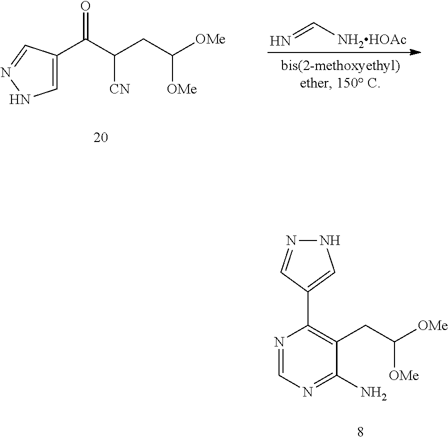

To a 4 mL glass vial with stir-bar was added 4,4-dimethoxy-2-(1H-pyrazole-4-carbonyl)butanenitrile 20 (45.9 mg), formamidine acetic acid salt (300 mg, 14 equiv), and bis(2-methyxyethyl) ether (0.500 mL). The vial was heated in a heater vial holder maintained at 150° C. for 1.5 hours while stirring, then cooled to room temperature. Sodium hydroxide solution (15 wt % in water, 1.0 mL) was charged to the vial. The vial was gently shaken for 5 minutes. Phosphate buffer (3 M phosphate, pH 7, 1 mL), bis(2-methyxyethyl) ether (0.500 mL), and activated charcoal (DARCO KB-G) were charged to the vial. The vial was gently shaken for 5 minutes, then filtered on a polypropylene filter to provide a clear, dark red organic layer and a clear, faintly yellow aqueous layer. The organic layer was purified by column chromatography (0 to 10% methanol in dichloromethane). The product-containing fractions were dried with a nitrogen stream and the residue was taken up in n-butanol (1.0 mL) and washed with tribasic potassium phosphate solution (1.0 mL, 1 molal). The organics were concentrated to provide 5-(2,2-dimethoxyethyl)-6-(1H-pyrazol-4-yl)pyrimidin-4-amine 8 (13.2 mg, 25.7% yield).

¹H-NMR (400 MHz, DMSO-d₆) δ 13.10 (s, 1H), 8.24 (s, 1H), 8.07 (br s, 1H), 7.95 (br s, 1H), 6.57 (s, 2H), 4.63 (t, J=5.5 Hz, 1H), 3.28 (s, 6H), 2.93 (d, J=5.5 Hz, 2H). ¹³C-NMR (101 MHz, DMSO) δ 163.44, 156.03, 155.75, 139.38 (br), 129.28 (br), 119.81, 107.80, 103.49, 53.88, 31.36.

LCMS (ESI, positive mode): Expected: 250.1 (M+H); Found: 250.1 (M+H).

### Example 6b: Preparation of 5-(2,2-Dimethoxyethyl)-6-(1H-pyrazol-4-yl)pyrimidin-4-amine (8)

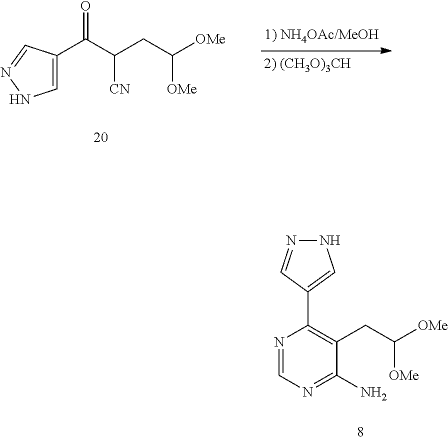 [SMILES: COC(CC(C#N)C(=O)c1cn[nH]c1)OC ; COC(Cc1c(N)ncnc1-c1cn[nH]c1)OC]

To a flask containing 4,4-dimethoxy-2-(1H-pyrazole-4-carbonyl)butanenitrile 20 (5 g) was added NH₄OAc (6.7 eq) in methanol (6 vol). The mixture was stirred overnight at 68° C., then methanol was removed by distillation and replaced by trimethylorthoformate. The mixture was heated to 92° C. and stirred for 4 hours, then was cooled to ˜0° C. and stirred for 2 hours, followed by filtration to remove solids. The filter cake was washed with acetonitrile (2×5 mL) and the resulting filtrate was concentrated in vacuo. Acetonitrile (15 mL) was added, and the resultant mixture was stirred at ambient temperature for 1.5 hours, followed by filtration to remove solids. The filter cake was washed with acetonitrile (5 mL) and the resulting filtrate was concentrated in vacuo to a brown liquid. The crude material was purified by silica-gel chromatography using 0-70% methanol/CH₂Cl₂ as eluent to give 8 as a light brown solid. QNMR (CD₃OD) indicated a molar yield of 65%.

### Example 7a: Preparation of (E)-2-(Amino(1H-pyrazol-4-yl)methylene)-4,4-dimethoxybutanenitrile (22)

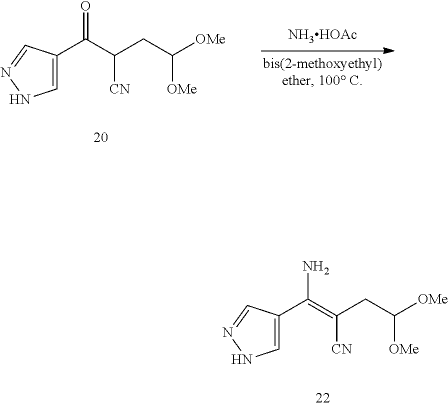 [SMILES: COC(CC(C#N)C(=O)c1cn[nH]c1)OC ; COC(C/C(C#N)=C(\N)c1cn[nH]c1)OC ; "NH3·HOAc"]

To a 4 mL glass vial was added 4,4-dimethoxy-2-(1H-pyrazole-4-carbonyl)butanenitrile 20 (161 mg), ammonium acetate (65 mg, 1.2 equiv), and bis(2-methyxyethyl) ether (2.0 mL). The mixture was warmed to 100° C. for 18 hours, then cooled to room temperature and transferred to a 20 mL scintillation vial. Methyl tert-butyl ether (3.0 mL) and tribasic potassium phosphate solution (3.0 mL, 0.5 M) were charged, and the mixture was aged for 1 hour. Solid potassium phosphate (0.40 g) and activated charcoal (DARCO KB-G, 0.115 g) were added and the vial was gently shaken. The mixture was filtered to provide a triphasic mixture. The top layer was removed to another vial. The two remaining layers were extracted with 5 mL methyl tert-butyl ether twice, and the three methyl tert-butyl ether layers were combined. The combined methyl tert-butyl ether extracts were dried with a nitrogen stream to provide (E)-2-(amino(1H-pyrazol-4-yl)methylene)-4,4-dimethoxybutanenitrile 22 containing 21.7 wt % bis(2-methyxyethyl) ether as a yellow oil (0.119 g, 58.5% yield).

¹H-NMR (400 MHz, Chloroform-d) δ 10.88 (s, 1H), 7.87 (s, 2H), 5.12 (s, 2H), 4.40 (t, J=5.1 Hz, 1H), 3.34 (s, 6H), 2.42 (d, J=5.1 Hz, 2H). ¹³C-NMR (101 MHz, CDCl₃) δ 151.55, 133.83, 124.62, 116.99, 105.51, 70.75, 54.74, 33.58.

LCMS (ESI, positive mode): Expected: 223.1 (M+H); Found: 223.1 (M+H).

### Example 7b: Preparation of (E)-2-(Amino(1H-pyrazol-4-yl)methylene)-4,4-dimethoxybutanenitrile (22)

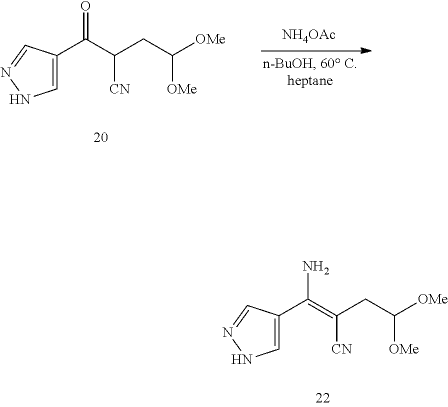 [SMILES: COC(CC(C#N)C(=O)c1cn[nH]c1)OC ; COC(C/C(C#N)=C(\N)c1cn[nH]c1)OC ; "NH4OAc"]

To a stirred solution of 20 (38.2 g, 171 mmol) in n-butanol (191 mL, 5 vol) was added ammonium acetate (66.0 g, 856 mmol, 5.0 equiv.). The resulting mixture was stirred at 60° C. for 15 hours then cooled to 20° C. A 0.5 M solution of dibasic potassium phosphate (191 mL, 5 vol) was added followed by n-butanol (76.4 mL, 2 vol). The agitation was then stopped and the aqueous layer removed. The organic layer was then washed with a 0.5 M solution of dibasic potassium phosphate (3×135 mL, 3×3.5 vol) then with 0.05 M dibasic potassium phosphate (153 mL, 3.5 vol). To the remaining organic solution was added carbon (Darco KB-G, 1.91 g) and the resulting suspension was stirred for 1 hour at 20° C. then filtered through Celite rinsing with n-butanol (76.4 mL, 2 vol). The combined filtrate was concentrated to a target of ˜2 vol and the resulting brown suspension was heated to 60° C. Heptane (131 mL, 3.4 vol) was added at 60° C. over a period of 2 hours then the resulting slurry was held at this temperature for one additional hour. After cooling to 20° C. the slurry was filtered under vacuum and rinsed with 25% n-butanol/heptane (67 mL, 1.75 vol). The filter cake was dried under vacuum at 50° C. to provide 22 as a tan powder (32.1 g, 84% yield).

### Example 7c: Preparation of (E)-2-(Amino(1H-pyrazol-4-yl)methylene)-4,4-dimethoxybutanenitrile (22)

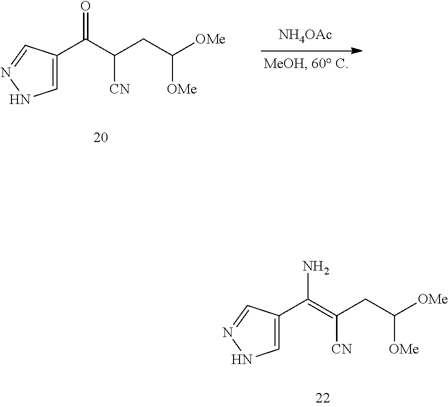 [SMILES: COC(CC(C#N)C(=O)c1cn[nH]c1)OC ; COC(C/C(C#N)=C(\N)c1cn[nH]c1)OC ; "NH4OAc"]

To a stirred solution of 20 (37.2 g, 167 mmol, 1.0 equiv.) in MeTHF (45.7% w/w solution) was added ammonium acetate (64.3 g, 5.0 equiv.) and methanol (186 mL, 5 vol). The resulting mixture was stirred at 60° C. for 22 hours then cooled to 20° C. To the mixture was added carbon (Darco KB-G, 1.86 g) and the resulting suspension was stirred for 1 hour at 20° C. then filtered through Celite rinsing with methanol (112 mL, 3 vol). The combined filtrate was concentrated to dryness to afford an amber clear oil which was cooled to 20-25° C. with agitation to afford a slurry. Water (223 mL, 6 vol) was added and the batch was agitated at 20-25° C. for 5 minutes. After cooling to 0-5° C., the mixture was stirred at this temperature for 2 hours. The slurry was filtered under vacuum and the filter cake was rinsed with water (112 mL, 3 vol). The filter cake was dried under vacuum at 50-60° C. to provide 22 as a beige solid (37.9 g, 100% yield-98.6% w/w by QNMR).

### Example 8: Preparation of 6-(1-Benzyl-1H-pyrazol-4-yl)-5-(2,2-dimethoxyethyl)pyrimidin-4-amine (8)

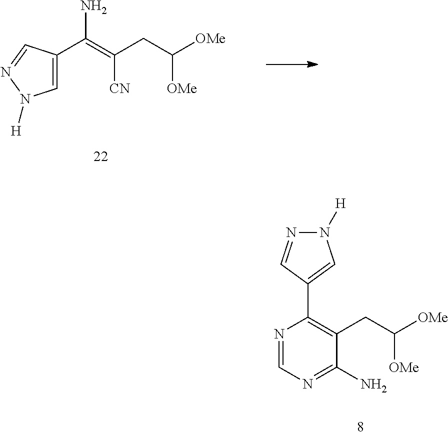 [SMILES: COC(C/C(C#N)=C(\N)c1cn[nH]c1)OC ; COC(Cc1c(N)ncnc1-c1cn[nH]c1)OC]

### Example 8a

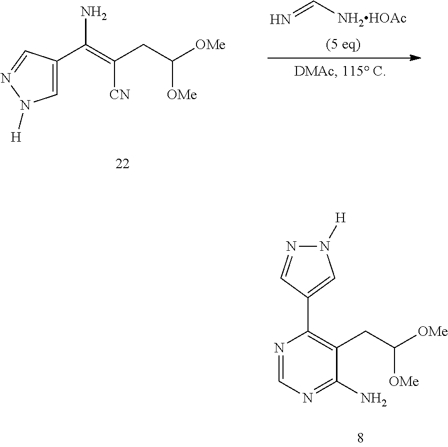

To a solution of 22 (5.0 g, 1.0 equiv.) in dimethylacetamide (DMAc) (20 mL) was added formamidine acetate (11.8 g, 5.2 equiv.) and the resulting suspension was heated to 115° C. After stirring at 115° C. for 36 hours the reaction was cooled to 90° C. and water (10 mL) was added. After stirring at 90° C. for one hour, the reaction was cooled to 20° C. and additional DMAc (10 mL) was added. The resulting dark solution was filtered through a pad of Celite which was subsequently rinsed with 3:1 DMAc/water (15 mL). The filtrates were combined, diluted with water (28 mL) and carbon (1.5 g) was added. The resulting suspension was stirred at 20° C. for one hour then filtered, rinsing through with 1:1 DMAc/water (7.5 mL). To the combined filtrate was added 25% w/w NaCl in water (4 mL) and the resulting mixture was extracted with 17% n-butanol/CH₂Cl₂ (4×24 mL). The organic layers were combined, washed with 15% w/w K₃PO₄ in water (15 mL), then concentrated under vacuum to ˜10 mL. DMAc (4 mL) was added to bring the total DMAc content to 10 mL, then the resulting solution was added to methyl tert-butyl ether (MTBE) (25 mL) precooled to −20° C. Intermediate 8 seed was added and the resulting slurry was stirred at −20° C. for 4 hours then additional MTBE (5 mL) was added. After stirring at −20° C. for an additional 4 hours, the slurry was filtered and the resulting cake was washed with 3:1 MTBE/DMAc (7.5 mL). After drying in a vacuum oven, 5-(2,2-dimethoxyethyl)-6-(1H-pyrazol-4-yl)pyrimidin-4-amine 8 was obtained as an off-white solid (2.91 g, 54% yield).

### Example 8b

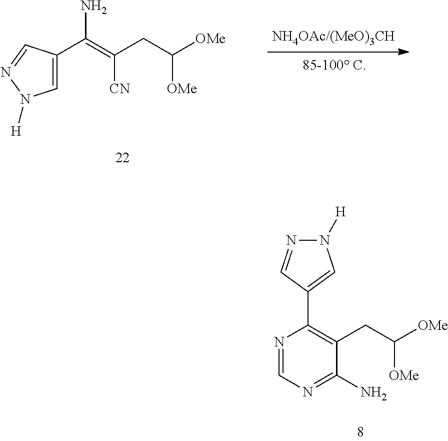 [SMILES: COC(C/C(C#N)=C(\N)c1cn[nH]c1)OC ; COC(Cc1c(N)ncnc1-c1cn[nH]c1)OC]

To a 100-mL, half-jacketed glass reactor with screw caps and magnetic stirrer was added solid pyrazole-enamine 22 (5.56 g, 25 mmol, 1.0 eq) and NH₄OAc (11.56 g, 150 mmol, 6.0 eq), followed by trimethyl orthoformate (TMOF) (50 mL, 9 V). The reactor was flushed with nitrogen and sealed tightly. The resulting slurry was stirred while the jacket's temperature was ramped up to 88° C. The slurry was dissolved resulting in a brown solution. The solution was stirred overnight (˜20 hours) while the jacket temperature was kept at 88° C. The mixture was cooled to 20° C., then was transferred to a round-bottom flask and was concentrated in vacuo to a brown liquid residue (17.9 g).

To a portion of this brown liquid (10.6 g, ˜61% of the original input) was added TMOF (9.0 mL) and NH₄OAc (7.04 g, 91.3 mmol). The mixture was heated in a sealed vial at 92° C. for 4 hours, then was cooled to −0° C. and stirred for 2 hours, followed by a filtration to remove solids. The wet filter cake was washed with acetonitrile (2×5 mL), the resulting filtrate was concentrated in vacuo, acetonitrile (15 mL) was added to the concentrate, the mixture was stirred at ambient temperature for 1.5 hours, followed by filtration to remove solid. The filter cake was washed with acetonitrile (5 mL) and the resulting filtrate was concentrated in vacuo to a brown liquid residue. The crude product was purified by silica-gel chromatography with 0-70% methanol/CH₂Cl₂ as eluent to yield 2.1 g of a light brown solid. ¹H-NMR (DMSO-d₆) confirmed the presence of product 8 and ˜8 w % acetic acid. Quantitative ¹H-NMR (CD₃OD) indicated 1.85 g of product 8, a molar yield of 49%.

### Example 8c

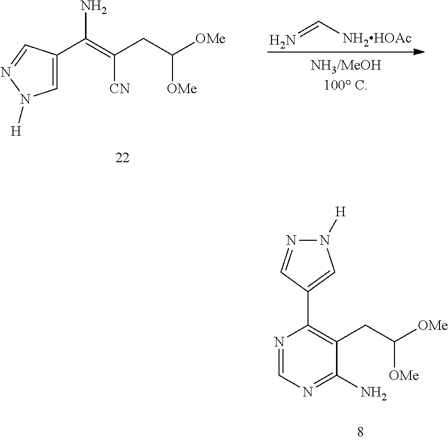

Pyrazole-enamine 22 (5 g) was mixed with formamidine acetate (14 g, 6.0 eq) and 7 N NH₃ in methanol (5 mL). The mixture was stirred and heated overnight in a sealed reactor with the jacket's temperature set at 120° C. Additional formamidine acetate (7 g, 3 eq) and 7 N NH₃ in methanol (5 mL) were added to the resultant reaction mixture and stirring was continued overnight with the jacket's temperature set at 120° C. The reaction mixture was concentrated in vacuo and the resulting residue was purified by a silica-gel plug with 10-100% methanol in acetonitrile as eluent to give 2.7 g of 8 as a light beige solid (Molar yield 48%).

### Example 8d

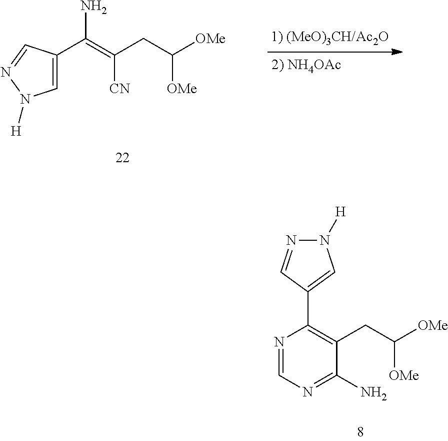 [SMILES: COC(C/C(C#N)=C(\N)c1cn[nH]c1)OC ; COC(Cc1c(N)ncnc1-c1cn[nH]c1)OC]

To a 100 mL reactor with overhead stirring was charged 22 (5.0 g), toluene (20 mL), TMOF (6 mL) and acetic anhydride (6 mL). The mixture was heated to 100° C. and stirred under nitrogen for approximately 16 hours. The jacket temperature was increased to 145° C., and approximately 15 mL of solution was distilled. To the resultant mixture was added toluene (15 mL), and the batch temperature was adjusted to 60° C. Ammonium acetate (8.8 g) was charged to the solution and the mixture was stirred under nitrogen for 5 hours. The batch temperature was adjusted to 20° C., and water (10 mL) was charged. The batch was cooled to 20° C., and the clear organic layer was discarded. Potassium phosphate solution (0.5 M, 80 mL) was added to the reactor. The reactor jacket was heated to 145° C. and 5 mL of solution was removed by distillation. The batch was cooled to 60° C., and approximately 2 mg of seed intermediate 8 was added. The mixture was cooled to −2° C., and another 2 mg of seed was added. The mixture was stirred at this temperature for 14 hours. The resultant suspension was filtered on a polypropylene filter funnel, then washed twice with cold water (10 mL×2). The tan, sand-like solid was suction-dried for 45 minutes yielding 3.9 g of solid which was shown by KF titration to be 33% water. Assay purity was determined to be 61% for a corrected yield of 41%. Identity of the sample was confirmed by HPLC, ¹H NMR, and mass spectrometry (ESI⁺ M+H: expected: 250.1, found: 250.1) comparison to an authentic sample.

### Example 8e

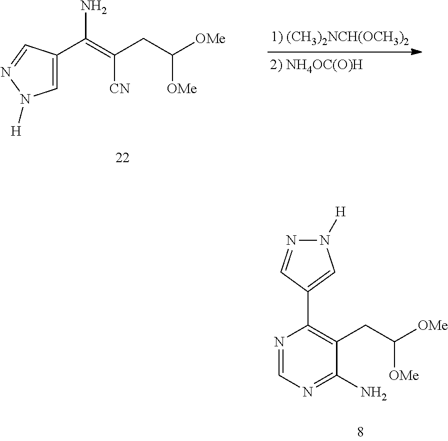 [SMILES: COC(C/C(C#N)=C(\N)c1cn[nH]c1)OC ; COC(Cc1c(N)ncnc1-c1cn[nH]c1)OC]

Procedure A: To a 20 mL scintillation vial equipped with a stir-bar was charged 22 (0.281 g), methanol (1.4 mL), and dimethylformamide dimethyl acetal (2.8 equiv). The vial was capped and the mixture was stirred for 4 hours in a vial holder warmed to 60° C. Ammonium formate (0.420 g) was charged to the vial, the vial was recapped and stirred at 60° C. for 23 hours. A stream of nitrogen was used to remove most of the methanol from the mixture. The mixture was cooled, and potassium phosphate solution (0.5 M, 2 mL) was charged to the residual oil. The resulting mixture was briefly stirred, additional potassium phosphate solution (1 M, 1 mL) was added and a white suspension immediately formed. This suspension was stirred for 5 minutes, then filtered. The vial and cake were washed with water (1 mL) twice, then the filter cake was washed three times with 1 mL methyl tert-butyl ether to facilitate removal of water. After 5 minutes of suction drying, the solids were transferred to a warmed (60° C.) vial and dried further with a nitrogen stream for 5 minutes to yield 0.132 g of a tan solid. This first crop was 72.1% w/w product by quantitative NMR and approximately 22% water by KF titration. The aqueous liquors were combined and cooled to 0° C. for 24 hours. A second, smaller crop was isolated by filtration and suction drying for 20 minutes to yield 0.018 g of a second crop at 91% w/w by quantitative NMR. Combined isolated yield of precipitated solids was 35%.

Procedure B: To the enamine 22 (2.0 g, 1.0 equiv.) was added 2-propanol (10 mL, 5 vol) and dimethylformamide dimethyl acetal (1.2 mL, 1.1 eq). The mixture was stirred at 80-85° C. for 2 hours then was partially cooled. Ammonium formate (1.75 g, 3.0 equiv.) was added to the mixture and the resulting mixture was and stirred at 80-85° C. for 20 hours. Potassium phosphate solution (0.5 M, 10 mL, 5 vol) was added to the mixture and the resulting mixture was concentrated at 85° C. to ˜5 vol before adding water (10 mL, 5 vol) and again concentrating at 85° C. to ˜5 vol. The mixture was cooled to 50° C. then a seed crystal of 8 (˜5 mg) was added. The mixture was cooled to 20° C., stirred for 1 hour, then filtered. The filter cake was washed with water (5 mL, 2.5 vol), then with MBTE (5 mL, 2.5 vol), then was dried under vacuum for 1 hour to yield 1.373 g of 8 as an off-white solid (93.0 wt %, assay purity was determined a corrected yield of 57%).

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A process for preparing a compound of Formula E: 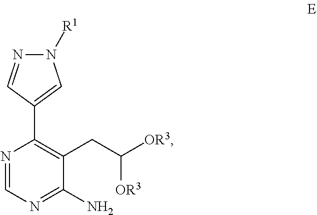
the process comprising the step of reacting a compound of Formula B: 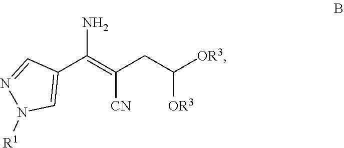
a compound of Formula C: 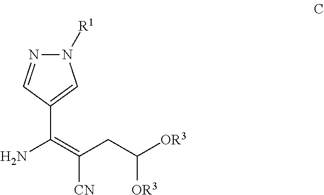
C or a mixture thereof
with formamidine or a salt thereof, or with a trialkyl orthoformate and an ammonium source, or with dimethylformamide dimethyl acetal and an ammonium source;
wherein R¹ is selected from H and a protecting group (PG), and wherein each R³ is C₁-C₁₀ alkyl, C₂-C₁₀ alkenyl, aryl, or the two R³s, taken together with the oxygen atoms to which they are attached, form a 5-7-membered heterocyclic ring which may optionally be substituted.

2. The process of claim 1, wherein R¹ is H.

3. The process of claim 1, wherein R¹ is a protecting group.

4. The process of claim 3, wherein R¹ is a protecting group which is benzyl.

5. The process of any one of claims 1-4, wherein R³ is methyl.

6. The process of any one of claims 1-4, wherein R³ is ethyl.

7. The process of any one of claims 1-6, wherein the process comprises reacting a compound of Formula B with formamidine or a salt thereof, or with a trialkyl orthoformate, or with dimethylformamide dimethyl acetal.

8. The process of any one of claims 1-6, wherein the process comprises reacting a compound of Formula C with formamidine or a salt thereof, or with a trialkyl orthoformate, or with dimethylformamide dimethyl acetal.

9. The process of any one of claims 1-8, wherein the step of reacting is performed in a protic solvent.

10. The process of claim 9, wherein the protic solvent is methanol or n-butanol.

11. The process of any one of claims 1-8, wherein the step of reacting is performed in an aprotic solvent.

12. The process of claim 11, wherein the aprotic solvent is toluene.

13. The process of any one of claims 1-12, wherein the process comprises the step of reacting a compound of Formula B with formamidine or a salt thereof.

14. The process of claim 13, wherein the formamidine or salt thereof is formamidine acetate.

15. The process of any one of claims 1-7 and 9-12, wherein the process comprises the step of reacting a compound of Formula B with ammonium acetate and trimethyl orthoformate.

16. A process for preparing a compound of Formula E: 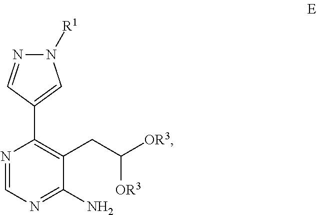
the process comprising the step of reacting a compound of Formula A: 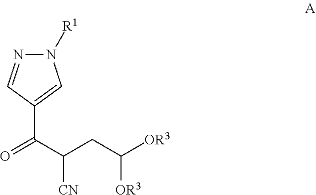
with formamidine or a salt thereof, or with an ammonium source and a trialkylorthoformate;
wherein R¹ is selected from H and a protecting group (PG), and wherein each R is C₁-C₁₀ alkyl, C₂-C₁₀ alkenyl, aryl, or the two R³s, taken together with the oxygen atoms to which they are attached, form a 5-7-membered heterocyclic ring which may optionally be substituted; and
the ammonium source is ammonia or an ammonium salt; provided that if each R³ is ethyl, R¹ is not H.

17. The process of claim 16, wherein R¹ is H.

18. The process of claim 16, wherein R¹ is a protecting group.

19. The process of claim 18, wherein R¹ is a protecting group which is benzyl.

20. The process of any one of claims 16-19, wherein R³ is methyl.

21. The process of any one of claims 16-19, wherein R³ is ethyl.

22. The process of claim 16, wherein the ammonium source is an ammonium salt.

23. The process of claim 22, wherein the ammonium salt is ammonium acetate.

24. The process of any one of claims 16-23, wherein the step of reacting is performed in an aprotic solvent.

25. The process of claim 24, wherein the aprotic solvent is bis(2-methyoxyethyl)ether.

26. The process of any one of claims 16-23, wherein the step of reacting is performed in a protic solvent.

27. The process of claim 26, wherein the protic solvent is methanol.

28. The process of any one of claims 16-27, wherein the process comprises the step of reacting a compound of Formula A with formamidine or a salt thereof.

29. The process of claim 28, wherein formamidine or salt thereof is formamidine acetate.

30. The process of any one of claims 16-28, wherein the process comprises the step of reacting a compound of Formula A with ammonium acetate and a trialkylorthoformate.

31. A process for preparing a compound of Formula 7: 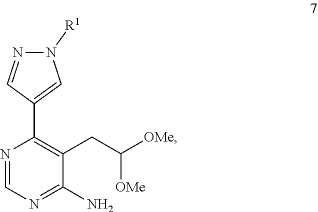
the process comprising the step of reacting a compound of Formula 6a: 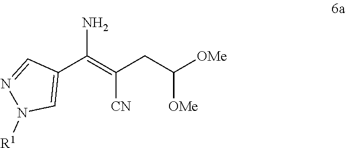
with formamidine or a salt thereof, or with trimethyl orthoformate, or with dimethylformamide dimethyl acetal;
wherein R¹ is selected from H or a protecting group (PG).

32. The process of claim 31, wherein R¹ is H.

33. The process of claim 31, wherein R¹ is a protecting group which is benzyl (Bn).

34. A process for preparing a compound of Formula 7: 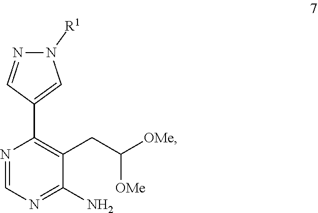
the process comprising the step of reacting a compound of Formula 5: 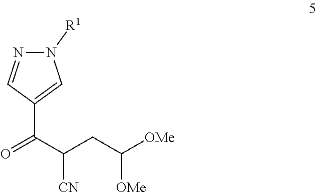
with formamidine or a salt thereof; or with an ammonium source and trialkyl orthoformate;
or with an ammonium source and dimethylformamide dimethylacetal;
wherein R¹ is selected from H or a protecting group (PG).

35. The process of claim 34, wherein R¹ is H.

36. The process of claim 34, wherein R¹ is a protecting group which is benzyl (Bn).

37. A compound represented by the structure: 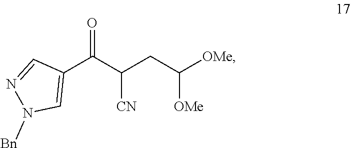 [SMILES: COC(CC(C#N)C(=O)c1cnn(Cc2ccccc2)c1)OC]
or a salt thereof
wherein Bn is a benzyl group.

38. A process for preparing a compound of Formula 5: 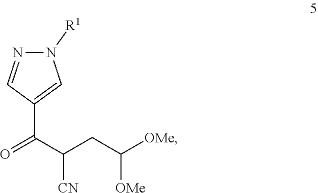
the process comprising the step of reacting a compound of Formula 1: 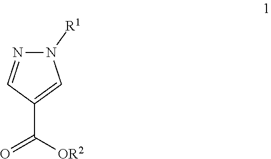
with Compound 4 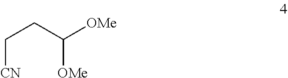
and a base;
wherein R¹ is selected from H or a protecting group (PG), and wherein R² is C₁-C₄ alkyl.

39. The process of claim 38, wherein R¹ is H.

40. The process of claim 38, wherein R¹ is a protecting group which is benzyl (Bn).

41. The process of any one of claims 38-40, wherein R² is methyl.

42. The process of any one of claims 38-40, wherein R² is ethyl.

43. The process of any one of claims 38-42, wherein the base is NaHMDS.

44. A process for preparing a compound of Formula 6a: 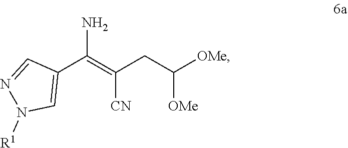
the process comprising the step of reacting a compound of Formula 5: 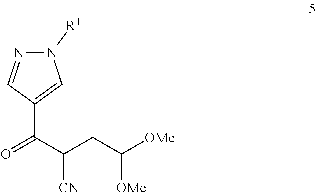
with an ammonium source;
wherein R¹ is selected from H or a protecting group (PG).

45. The process of claim 44, wherein R¹ is H.

46. The process of claim 44, wherein R¹ is a protecting group which is benzyl (Bn).

47. The process of any one of claims 44-46, wherein the ammonium source is selected from ammonium formate, ammonium chloride and ammonium acetate.

48. A compound represented by the structure: 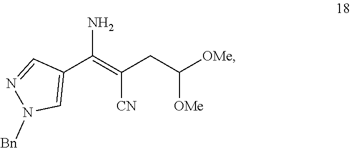 [SMILES: COC(C/C(C#N)=C(\N)c1cnn(Cc2ccccc2)c1)OC]
or a salt thereof
wherein Bn is a benzyl group.

49. A compound represented by the structure: 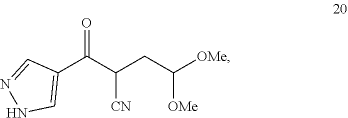 [SMILES: COC(CC(C#N)C(=O)c1cn[nH]c1)OC]
or a salt thereof.

50. A compound represented by the structure: 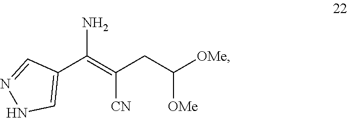 [SMILES: COC(C/C(C#N)=C(\N)c1cn[nH]c1)OC]
or a salt thereof.

51. A compound represented by the structure: 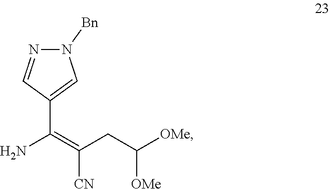 [SMILES: COC(C/C(C#N)=C(/N)c1cnn(Cc2ccccc2)c1)OC]
or a salt thereof
wherein Bn is a benzyl group.

52. A compound represented by the structure: 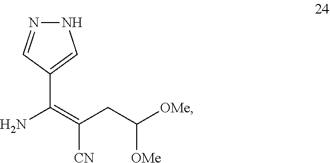 [SMILES: COC(C/C(C#N)=C(/N)c1cn[nH]c1)OC]
or a salt thereof.
